(19) United States
(12) Patent Application Publication
(10) Pub. No.: US 2021/0345955 A1
(43) Pub. Date: Nov. 11, 2021

(54) PORTABLE REAL-TIME MEDICAL DIAGNOSTIC DEVICE

(71) Applicant: DAVE ENGINEERING LLC, Chesterfield, MO (US)

(72) Inventor: DAVE JONES, ST. CHARLES, MO (US)

(21) Appl. No.: 17/308,532

(22) Filed: May 5, 2021

Related U.S. Application Data
(60) Provisional application No. 63/020,315, filed on May 5, 2020.

(51) Int. Cl. A61B 5/00; G06T 7/13; G06T 7/11

(52) U.S. Cl. CPC A61B 5/4842; A61B 5/6801; A61B 5/742; G06T 7/13; G06T 2207/30096; G06T 2207/20081; G06T 2207/20084; G06T 2207/20076; G06T 7/11

(57) ABSTRACT

A portable device for providing real-time medical ailment and cancer diagnosis. The portable device captures image information in a particular location for use to diagnose and update prior diagnosis of such medical ailments. The device captures new images, segments the images based on known or existing artificial intelligence, or image processing algorithms to isolate the region of interest from the image, process the region of interest via a learned diagnostic model, and provide output about the region of interest in real time. The output information contains a set of diagnostic probabilities in real time. The device communicates within a platform for receiving the generated information and applying deep learning or machine learning algorithms to update the diagnostic model and to trend data about the image with previous snapshots. The updated learned diagnostic model can then be downloaded to the device for further diagnoses.

### CROSS-REFERENCE TO RELATED APPLICATIONS

This application claims priority to U.S. App. No. 63/020,315 filed May 5, 2020, which is entitled “PORTABLE REAL-TIME MEDICAL DIAGNOSTIC DEVICE” and which is incorporated herein by reference.

### STATEMENT REGARDING FEDERALLY SPONSORED RESEARCH OR DEVELOPMENT

Not Applicable.

### BACKGROUND

In the medical field, a medical ailment can often times take days to years to arrive at a correct diagnosis. More well-known methods of correctly diagnosing a medical ailment can range from getting an X-ray or a CT-scan to more complex and drawn out clinical trials, which can often take several years.

More recent research has been focused on utilizing hardware and software in the loop applications, in an attempt to diagnose medical ailments more quickly. More well-known applications of hardware/software in loop diagnostic methods are found in ultrasound, heart rate monitors, and other systems that have been in use for several years. These systems are based on early hardware/software combinations in the loop capabilities, which typically do not include high performance computing capabilities, or are not capable of providing real-time decisions to medical personnel.

In the fields of artificial intelligence and machine learning, complex algorithms that are configured to receive, process, and learn statistical parameters associated with input data are becoming more prevalent. Recent applications are relying on input images and other biomarkers as snapshots in time. These algorithms are configured with the statistical learning capabilities to receive, analyze, build trends, and make predictions based on large sample data sets with known or otherwise predicted outcomes.

However, successfully implementing these algorithms has typically required excessive power, high performance computing, specialized hypercore processing, significant memory storage, and a networked data supply, thereby limiting their application essentially to only large and very complex systems. Where these parameters are met, artificial intelligence (AI) as applied to correctly diagnosing medical ailments, including certain types of cancer based on locational biased data or image segmentation, has shown some promise. However, it remains generally impractical when situations arise where such systems are not readily available. A need for a more portable, low power, self-contained, high performance computing, diagnostic capability is currently needed for real-time medical diagnoses based on image segmentation and locational biased data. In particular, a need for the capability to capture and trend this input data for making real-time cancer diagnoses is needed.

In the following sections of this disclosure, a system, device, and method thereof that overcomes such shortcomings of prior art devices, systems and methods are disclosed.

### BRIEF SUMMARY

A mobile handheld imaging AI device which supports local real time classification object detection and image segmentation is provided for herein. This evaluation tool delivers the probability of like results prior to a doctor patient interaction. Benefits of this disclosure include an object classification and segmentation method for capturing and trending in real-time cancer images to generate a quicker cancer type, diagnosis, reduction in patient visits, reduced duration until proper care is administered, and the opportunity for in-home remote care. The platform is data model, retrainable for multi-use portable diagnostics.

The system may encompass any means of providing portable medical diagnostics, which may include a handheld mobile battery-operated device, or a device that connects to a mobile phone or computer device. The key components of the device include at least one tensor processing unit (TPU) or comparable edge inference device for image processing, image segmentation, machine learning, or artificial intelligence in addition to a core processor and memory. The device may have a camera, a display, connectivity means; or connect via well-known traditional methods to a device that has these features onboard, such as a mobile phone or computer. The device or mobile phone contributes locational Global National Satellite Systems/Global Positioning Systems (GNSS/GPS) data for use in locational out break and prevention.

The process workflow allows testing and validation of the objective data in stages within clinical research outputting a tensor flow model out to the field as bio marker data sets. The bio marker data sets include image information about a medical ailment, such as cancer, and provides this image information back to the research level. The device transmits the image and usage data back to the research level for further analysis. The analysis may reveal an early cancer detection or may lead to identification of a particular type of cancer or diagnosis. Furthermore, the system may use previous image data to generate a trended pattern of growth, remission, or other type of diagnosis. The analysis will utilize the new input images to update the learned diagnosis model. The updated model can be transmitted back to the remote devices to provide further diagnosis information, thereby closing the loop. The result of the workflow is a real-time trended diagnosis for cancer or similar diseases, and the trending over time of them.

These features and other features of the present disclosure will be discussed in further detail in the following detailed description.

### BRIEF DESCRIPTION OF THE DRAWINGS

FIG. 1 discloses a conceptual connectivity model of an embodiment of the disclosure.

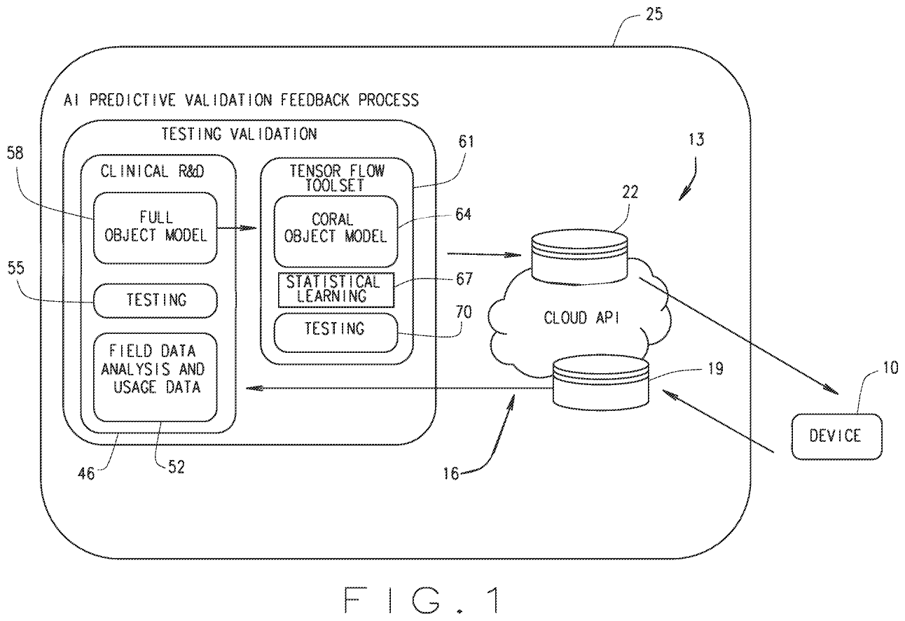

FIG. 2 shows an objective view of a device that is capable of satisfying a preferred aspect of the present disclosure.

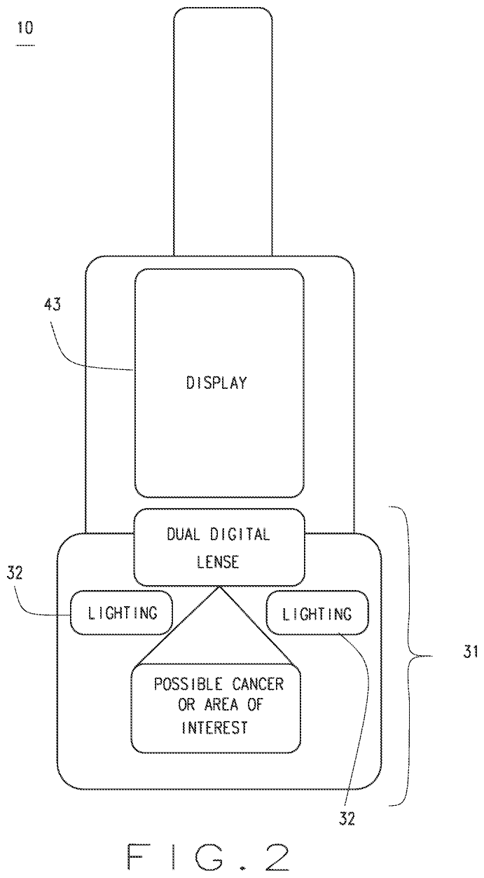

FIG. 3 shows an objective view of an embodiment of the computing hardware of the disclosure.

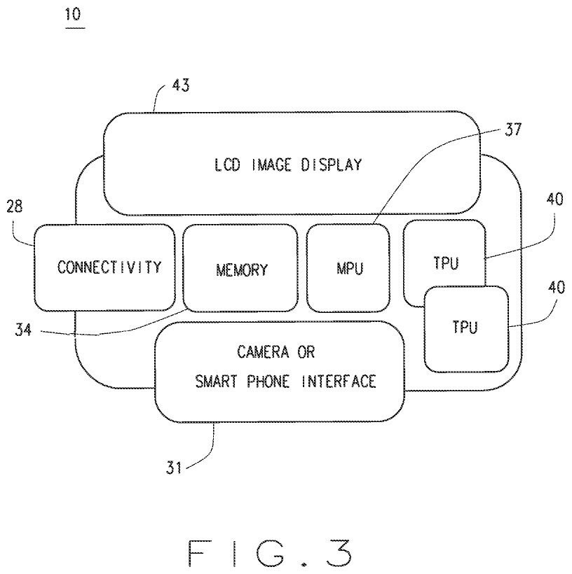

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what I presently believe is the best mode of carrying out the claimed invention. Additionally, it is to be understood that the claimed invention is not limited in its application to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

A preferred embodiment of the disclosure discloses a device 10 that is configured for wirelessly connecting to a communication network 13 for collecting, transmitting, and receiving diagnostic information from a cloud system 16. The cloud system 16 comprises a first database 19 that is configured to receive the inbound diagnostic information from the device 10 when it is connected to the wireless communication network 13. The cloud system 16 further comprises a second database 22 that is configured to transmit diagnostic model information to the device 10 when it is connected to the communications network 13. The first database 19 and the second database 22 may comprise a single computer system 25 that is configured to store each of the first database 19 and second database 22 in memory, or it may comprise multiple separate computer systems that are configured to receive, process, and transmit the diagnostic image and modeling information separately between the device 10 separately. One such application of this system is for making real-time cancer diagnoses based on past images, image segmentation, and trended information from previous snapshots.

The device 10 may be a handheld device, a wearable device, or another type of portable device that is capable of connecting with a communication network, capturing and storing input information, storing a learned diagnostic model on an internal memory, processing the input information with the stored diagnostic model, and generating a set of diagnostic information based on the results of processing the captured input information with the stored diagnostic model. The device 10, which maybe a mobile phone or handheld device includes a locational system, such as GPS, GNSS, or the like for providing a location associated with the data that is provided.

The network connectivity capability 28 on the device 10 may be a cellular network antenna, a Wi-Fi antenna, USB port, ethernet connector, or another means of providing network connectivity. The network connectivity capability 28 is configured both to transmit generated diagnostic information to the first database 19 on the cloud system 16 and to receive diagnostic model information from the second database 22 on the cloud system 16.

The device comprises an image capture capability 31, which is typically a camera, but may also be a form of infrared, ultraviolet, X-Ray, or the like. The image capture capability 31 is configured for capturing high resolution images of the diagnostic target, on an order of magnitude using techniques and equipment known to those of skill in this art appropriate for any particular application. The image capture capability may include a rapid capture or a video stream capability for capturing multiple high-resolution images for trending the frames that are processed by the learned diagnostic model.

The image capture capability 31 further comprises a plurality of illumination elements 32 that are configured to illuminate the targeted area for image capture. The illumination elements 32 may be set by the user to vary the illumination intensity based on the presence of external light.

The image capture capability 31 further comprises a dual digital lens 33 that is capable of capturing the input image data as previously stated at a high resolution required for performing real-time medical diagnostics in the field. The internal processing software is configured to receive the input image data in high resolution and providing an image segmentation capability that can isolate regions of interest in the image. The regions of interest may include analyzing images that reveal a multitude of types of cancer by phase and location.

The device 10 comprises an internal memory module 34 configured to store a learned diagnostic model as well as captured input image information from the image capture capability 31 that is capable of being processed by the learned diagnostic model. The memory module 34 may be of a solid-state device type, or the like, capable of high-performance data retrieval access time for facilitating fast processing in near real-time at a relatively low power. The memory module 34 has capacity sufficiently large enough to store both the learned diagnostic model, as well as multiple captured input images for processing with the learned diagnostic model.

The device 10 comprises a microprocessor unit (MPU) 37 that is configured to control the diagnostic process. The MPU 37 is configured to facilitate a wide variety of operations required by the device 10, including receiving, storing, and retrieving the learned diagnostic model from the network connectivity capability into and from memory, receiving, storing, and retrieving the stored image information from the image capture capability into and from memory, storing the retrieved diagnostic model onto a high-performance processing unit 40, storing the retrieved image information onto the high-performance unit 40 for processing by the learned model, and facilitating external communication through the network connectivity module 28 to the communication network 13. The MPU 37 may be a standard microprocessor type comprising designed for portable devices.

The high-performance processing unit 40 may be of the graphics processing unit (GPU) type, a tensor processing unit (TPU), or other comparative edge inference unit, herein after referred to as TPU. The TPU 40 is a processing unit with similarities to the MPU 37 but has an added configuration of being able to execute many computations in parallel, thereby providing for high-speed real-time data processing. This capability is necessary for performing real-time medical diagnostic computations on the captured image information by the learned diagnostic model.

The device 10 comprises an image display region 43 that is capable of displaying the captured image information from the image capture capability 31. This image display region 43 may be an LCD display, or the like and can display a variety of image types, which may include x-rays, or the like. The image display region 43 may also be comprised of a touch screen, thereby providing a means for the user to interact with the system by scrolling or panning the captured image for observing the area of interest more closely. With this feature, the user can zoom in, rotate, focus, denoise, and trend an area of interest to provide a predictive diagnostic output that may be transmitted to a qualified medical professional.

Upon capturing an input image or video stream in an area of interest, image processing, artificial intelligence, machine learning, and deep learning predictive analytics can be applied. From stored memory, the learned diagnostic model can be transmitted from the MPU 37 to the TPU 40 where it is loaded for high-performance data processing. Through the parallel computational processing capability of the TPU 40, as previously mentioned, the diagnostic model is capable of receiving the captured input information and generating an output information stream that may be indicative of an appropriate medical diagnosis from the learned model.

The generated output information will include a set of probabilities, images, and past trends in the image. The model is configured to provide additional information based on the generated output information and can be used to make real-time medical diagnoses. In particular, the isolated region of interest from the original input image information may be linked to previous images of interest through the learned diagnostic model. The linked images form a trend of progression on the information in the images and can be used for early cancer detection, to identify a variety of types of cancer based on location, or can be used to administer a new diagnosis or to provide real-time updates on a previous cancer diagnosis. With this capability, oncologists making use of the system can provide treatment recommendations or prescribe new types of treatment to patients without requiring an in person visit or an appointment.

Upon generation of the output information stream, that may include a set of probabilities associated with learned diagnoses or other means for providing predictive diagnoses, captured input information and the output information stream are processed by the MPU 37 to transmit from portable device 10 through the connected network capability 28 to the first database 19 for processing by the computer system 25.

Once the information package consisting of the generated output information and the captured input image is received by the first database 19, it is transmitted to a clinical research and analysis system 46 where testing and validation of the data is allowed to take place by a testing and validation module 49.

The data that is received from the device 10 that comprises the captured input image data and the generated results after the learned diagnostic model processes the input data comprises a set of predicted bio marker data that indicate the diagnostic results. The clinical research and analysis system 46 reviews this data as it is received from the field data analysis and usage module 52. The data use and analysis module processes the data to be converted into a useable form for data testing by a testing module 55. The testing module 55 may use image processing techniques or other pattern recognition techniques, such as edge detection, corner detection, or neural network connectivity models, such that the test results reveal detailed bio marker data sets associated with the captured input image, as well as trends of images to correlate and assist in pandemic outbreak tracking by providing geographical coordinate location of the image data capture.

The results of the testing module 55 are transmitted to an object generation model 58, where the bio marker data is processed to generate a bio marker model of the input image data. The bio marker model can then be further processed and matched with similar bio marker models for generating a trended pattern. These trended patterns then become readily available for insertion into an updated model generation module 61, which receives the trended bio marker model patterns as input data for generating an updated learned diagnostic model. The bio marker model patterns are used as inputs to the pattern recognition, machine learning, artificial intelligence, or deep learning model generation module 61 for refining the learned diagnostic model patterns. This process may be repeated as many times as necessary to continue refining the diagnostic model precision.

The model generation module 61 of the clinical research and analysis system 46 is configured to be capable of high-performance computing. It may be built on a tensor flow toolset, a Python toolset, or the like for making use of the parallel data processing capabilities associated with high-performance computing capabilities.

The model generation module 61 comprises two major sub modules that are responsible for receiving the trended bio marker model patterns and updating the learned diagnostic model. First, the coral object model 64 receives the trended bio marker model patterns and analyzes them against the current iteration of the learned diagnostic mode. The analysis may comprise a residual differentiation, a mean absolute deviation, or the like for determining the difference between the trended bio marker model patterns and the current model. The coral object model 64 will utilize the generated residual model as input to a statistical learning module 67. The statistical learning module will receive the residual model, and use an optimization and refinement algorithm for updating the learned diagnostic model. The trended bio marker model patterns are then processed by the updated learned diagnostic model to generate a new set of residual models. This process continues for a predefined number of iterations until the updated model has sufficiently learned the trended bio markers that were captured in the original input image data from the device 10.

Once the learned diagnostic model has been updated, a second testing module 70 in the model generation module 61 will use the newly generated diagnostic model to test its performance against known previously captured diagnoses. The testing process is configured to identify new differences that have been introduced as a result of updating the diagnostic learned model by learning the trended bio marker models from the testing and validation module 49. This process continues until the updated diagnostic model and the results of testing the updated model against previously captured diagnoses begins to converge on a minimum residual error. When this occurs, the updated learned diagnostic model is stored in memory for further refinement when a new set of input image information and generated results are used to generate another trended bio marker model pattern.

When the learning process is completed, the new learned diagnostic model is transmitted along the communication network 13 to the second database 22. The device 10 is in network connectivity with the second database and is triggered when a new diagnostic model becomes available. When this happens, the new diagnostic model is downloaded to the devices for further diagnostic processing.

The new learned diagnostic model is updated based on the input images captured by the device 10 as it is deployed in the field. The devices capture a wide variety of images that display certain types of cancer in various stages of progression. The learned diagnostic model, as it is deployed on the device 10, in conjunction with the onboard image processing software, is capable of receiving new images from the input image capture capability, segmenting the images to isolate the regions of interest, processing the regions of interest by the updated learned diagnostic model, updating the output information to provide a real-time cancer diagnosis based on type and progression, transmitting the data to the cloud system 16 for updating the model, storing the model in the cloud system 16 learning from the trended image, and transmitting a new model to the remote device 10. With this capability, the cloud system 16 and device 10 are configured to work together to capture images in real time, process the images in real time, provide an identification or updated diagnosis in real time, and transmit the generated information to update the model in real time.

In view of the above, it will be seen that the several objects and advantages of the present invention have been achieved and other advantageous results have been obtained.

As various changes could be made in the above constructions without departing from the scope of the invention, it is intended that all matter contained in the above description or shown in the accompanying drawings shall be interpreted as illustrative and not in a limiting sense.

What is claimed is:

1. A portable real-time medical diagnostic device comprising a mobile unit,
a high-performance processing unit within or connected to the mobile unit configured for localized edge AI object detection and image segmentation of bio marker datasets updatable in the field,
a microprocessor unit,
a memory module,
an image or other sensory capture component,
a display,
and a network connectivity module.

2. The portable real-time medical diagnostic device of claim 1 wherein the high-performance processing unit, the memory unit and the display are configured to combine to provide real time display of like-match probability outcomes for early detection and progress tracking of a disease detectable by tracking bio marker datasets updatable in the field.

3. The portable real-time medical diagnostic device of claim 2 wherein the detectable disease is a cancer.

4. The portable real-time medical diagnostic device of claim 2 wherein the tracking of bio marker datasets comprises an analysis of past images, image segmentation and trended information

5. The portable real-time medical diagnostic device of claim 1 wherein the mobile device is dimensioned to be hand-held or configured to be wearable.

6. The portable real-time medical diagnostic device of claim 1 wherein the device is configured to capture and store input information, store a learned diagnostic model in the memory, and use the high-performance processing unit to process the input information with the stored diagnostic model to generate the bio marker datasets to provide a set of diagnostic information based upon the processing.

7. The portable real-time medical diagnostic device of claim 1 wherein the device further comprises a locational system.

8. The portable real-time medical diagnostic device of claim 1 wherein the image or other sensory capture component is selected from the group consisting of a visual light, infrared, ultraviolet and X-ray spectrum capture component.

9. The portable real-time medical diagnostic device of claim 1 wherein the display further comprises a touch screen.

10. The portable real-time medical diagnostic device of claim 1 further comprising a dual digital lens.

11. A platform for collecting and analyzing locational biased disease data using object detection and image segmentation yielding statistical probability and location correlation useful for analyzing cancer types by location wherein the platform comprises a portable real-time medical diagnostic device comprising a mobile unit,
a high-performance processing unit within or connected to the mobile unit configured for localized edge AI object detection and image segmentation of bio marker datasets updatable in the field,
a microprocessor unit,
a memory module,
an image or other sensory capture component,
a display,
and a network connectivity module, and
a communication network configured to collect, transmit and receive diagnostic information from a first database configured to receive the inbound diagnostic information from the device and a second database configured to transmit diagnostic model information to the device when it is connected to the communication network.

12. The platform of claim 11 configured for transference of trained machine learning bio marker data sets, where the medical diagnostic device comprises a tensor flow device deployable in the field that does not require training for medical diagnostic performance.

13. A process configured for transference of trained machine learning bio marker data sets, wherein a portable real-time medical diagnostic device comprising a mobile unit,
an image or other sensory capture component,
and a high-performance processing unit within or connected to the mobile unit configured for localized edge AI object detection and image segmentation of bio marker datasets updatable in the field, does not require training for medical diagnostic performance in the field.
